(19) United States
(12) Patent Application Publication
(10) Pub. No.: US 2025/0152208 A1
(43) Pub. Date: May 15, 2025

(54) INTERSPINOUS-INTRASPINOUS PROSTHESIS

(71) Applicant: Francesco Maria LO BIANCO, Filignano (Isernia) (IT)

(72) Inventor: Francesco Maria LO BIANCO, Filignano (Isernia) (IT)

(21) Appl. No.: 18/715,464

(22) PCT Filed: Nov. 17, 2022
(86) PCT No.: PCT/EP2022/082215

§ 371 Date: May 31, 2024

(30) Foreign Application Priority Data
Dec. 2, 2021 (IT) 102021000030545

(51) Int. Cl. A61B 17/70

(52) U.S. Cl. CPC A61B 17/7068

(57) ABSTRACT

An interspinous-intraspinous prosthesis comprising a clamp constituted by a first, fixed element provided with at least one protruding tab and by a second, movable element, which can move along the fixed element and is provided with at least one protruding contrast tab. Removable mutual locking elements are interposed between the first, fixed element and the second, movable element.

At least one component chosen between the tab and the protruding contrast tab comprises at least one through hole for the accommodation of at least one screw designed to be tightened on a respective spinous process of a vertebral body clamped between the tab and the protruding contrast tab.

The present invention relates to an interspinous-intraspinous prosthesis, i.e. a surgical medical device that can be implanted between contiguous vertebrae of the spinal column of an individual suffering from specific conditions, which constitutes a key element in all spinal surgery.

The use of interspinous prostheses involves a contained surgical invasiveness, with minimum muscular damage, which results in an easier post-operative period for the patient, with respect to the application of traditional screwed plates.

This aspect is particularly relevant, in particular with that category of patients (the elderly, patients affected by motor or metabolic difficulties, etc.) who cannot carry out a complete and effective rehabilitative therapy.

The prosthesis interspinous is designed for spinal stabilization via a rigid connection between two vertebral bodies with development of arthrodesis.

The prosthesis is normally clamped on the spinous processes of contiguous vertebrae: the clamping of these protuberances determines the rigid connection of the treated bodies. Immediately after the installation, the locking is ensured solely by the quality of the clamping on the spinous processes, and therefore the patient must necessarily limit their movements in order to facilitate a good radication, until the formation of a bone tissue to cover the prosthesis completes the consolidation with arthrodesis.

The time required for correct consolidation is not the same for all patients and therefore it is necessary to limit the patient's movements for periods that vary from patient to patient, in order to ensure the good outcome of the operation. By contrast, however, some patients cannot be immobilized owing to previous diseases.

The aim of the present invention is to solve the above mentioned drawbacks by providing an interspinous-intraspinous prosthesis that offers high stability right from the moment of its implantation.

Within this aim, an object of the invention is to provide an interspinous-intraspinous prosthesis that enables the patient to not require a period of immobility after the operation to implant the prosthesis.

Another object of the invention is to provide an interspinous-intraspinous prosthesis with a space occupation corresponding entirely to that of traditional prostheses.

Another object of the invention is to provide an interspinous-intraspinous prosthesis that can be easily implanted with operating techniques substantially similar to those in use for conventional prostheses.

Another object of the invention is to provide an interspinous-intraspinous prosthesis that is adapted to ensure the necessary formation of bone tissue within substantially contained times.

Another object of the present invention is to provide an interspinous-intraspinous prosthesis that is of low cost, easily and practically implemented, and safe in use.

This aim and these and other objects that will become more apparent hereinafter are achieved by an interspinous-intraspinous prosthesis of the type comprising a clamp constituted by a first, fixed element provided with at least one protruding tab and by a second, movable element, which can move along said fixed element and is provided with at least one protruding contrast tab, removable mutual locking means being interposed between said first, fixed element and said second, movable element, characterized in that at least one component chosen between said tab and said protruding contrast tab comprises at least one through hole for the accommodation of at least one screw designed to be tightened on a respective spinous process of a vertebral body clamped between said tab and said protruding contrast tab.

Further characteristics and advantages of the invention will become better apparent from the detailed description that follows of a preferred, but not exclusive, embodiment of the interspinous-intraspinous prosthesis, which is illustrated by way of non-limiting example in the accompanying drawings wherein:

FIG. 1 is a partially exploded schematic perspective view of a possible embodiment of an interspinous-intraspinous prosthesis according to the invention, from a first point of view;

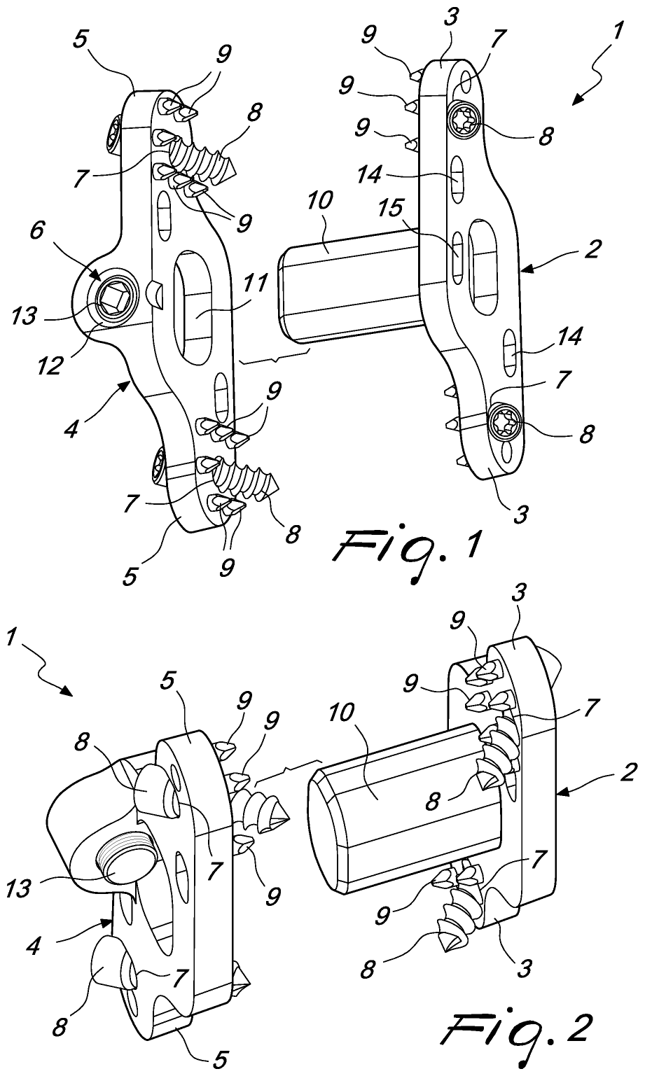

FIG. 2 is a partially exploded schematic perspective view of the interspinous-intraspinous prosthesis of FIG. 1 from a second point of view;

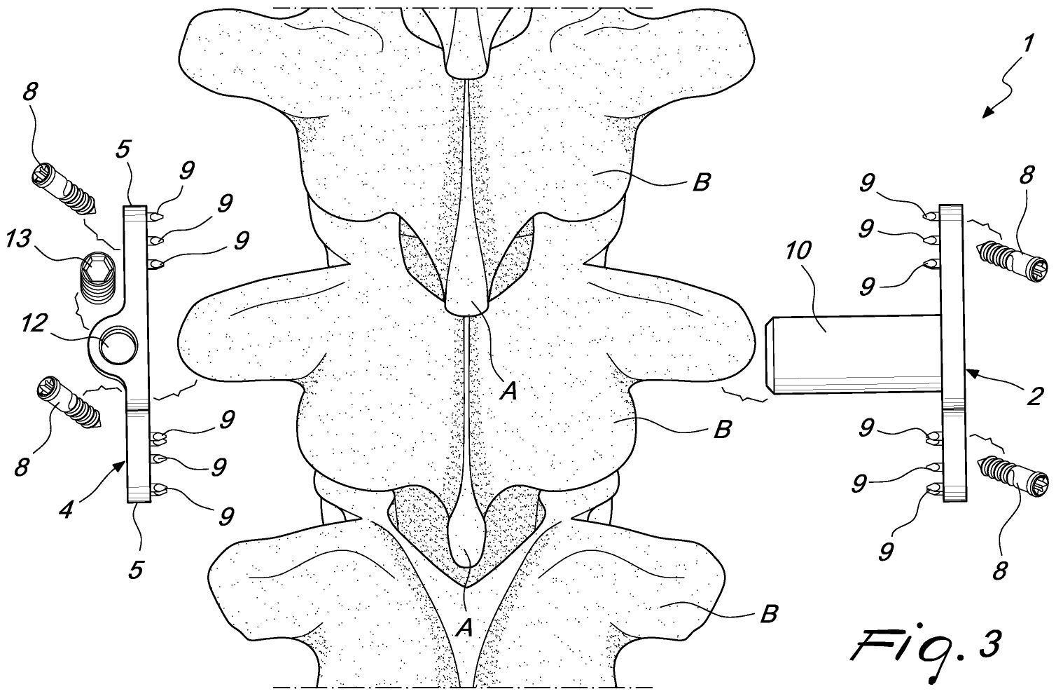

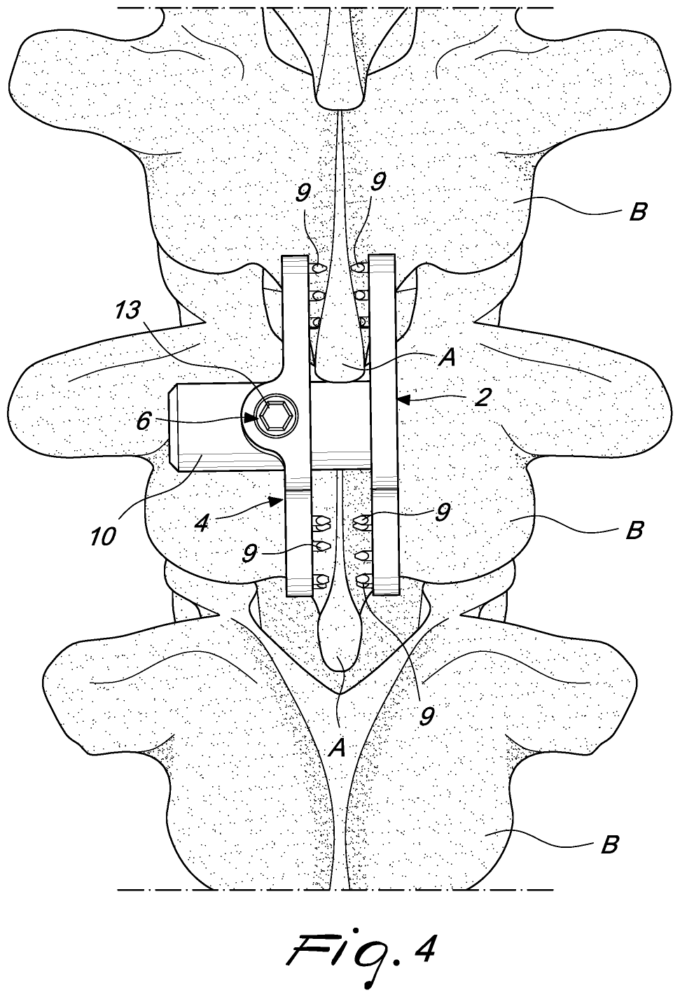

FIG. 3 is an exploded schematic front elevation view of a step preliminary to the implantation of the interspinous-intraspinous prosthesis of FIG. 1;

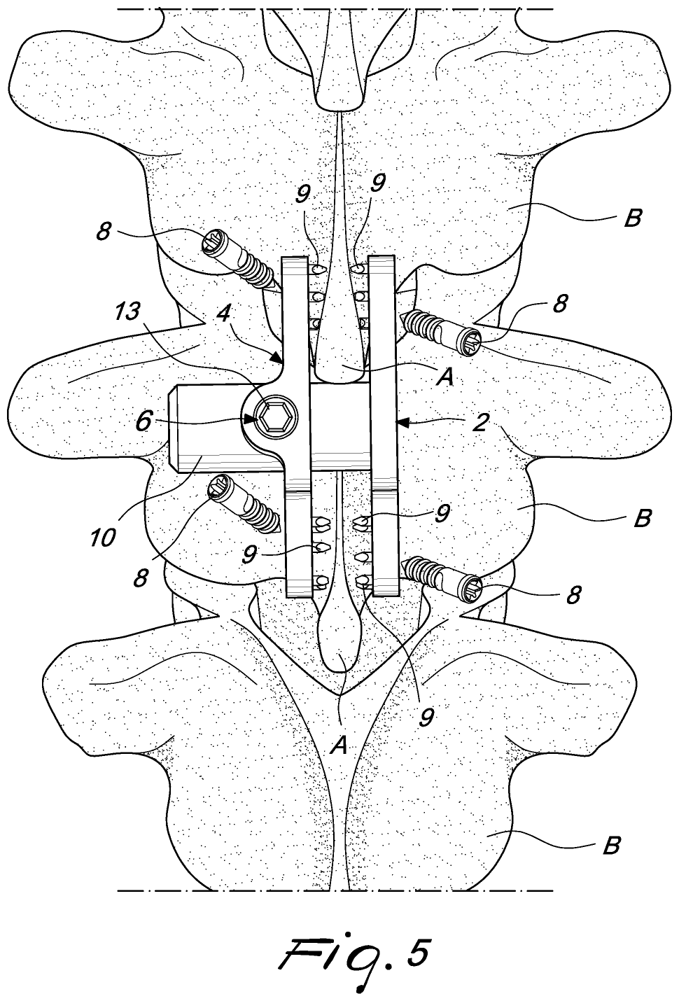

FIG. 4 is a schematic front elevation view of a second step, of clamping of the spinous processes of two contiguous vertebral discs by means of the prosthesis of FIG. 1;

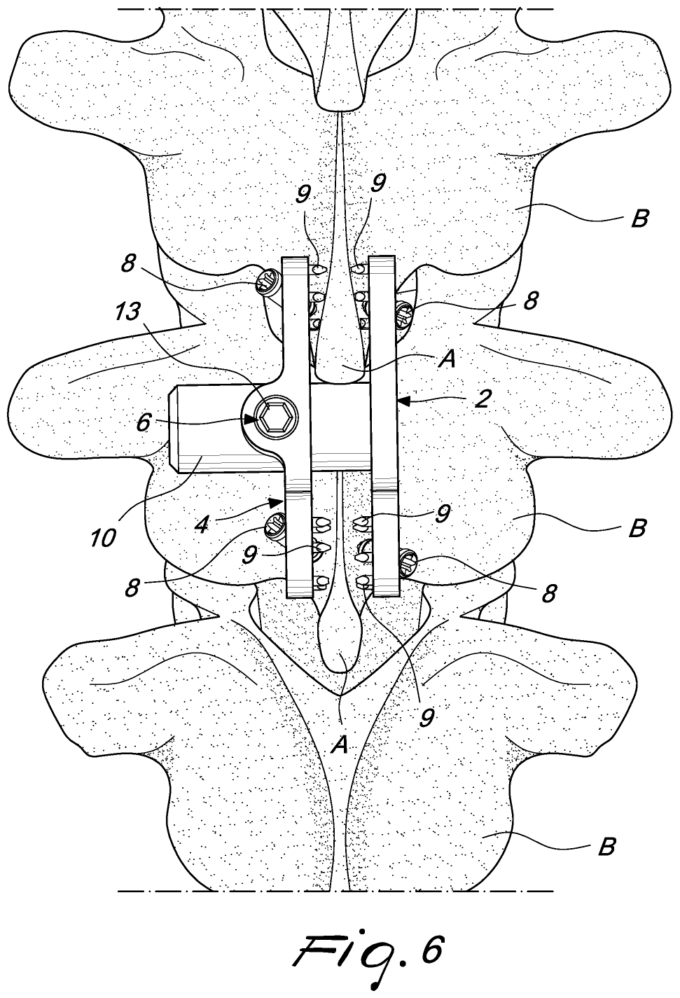

FIG. 5 is a schematic front elevation view of a third step, of inserting screws into the elements of the prosthesis which was previously arranged to clamp the spinous processes in the second step;

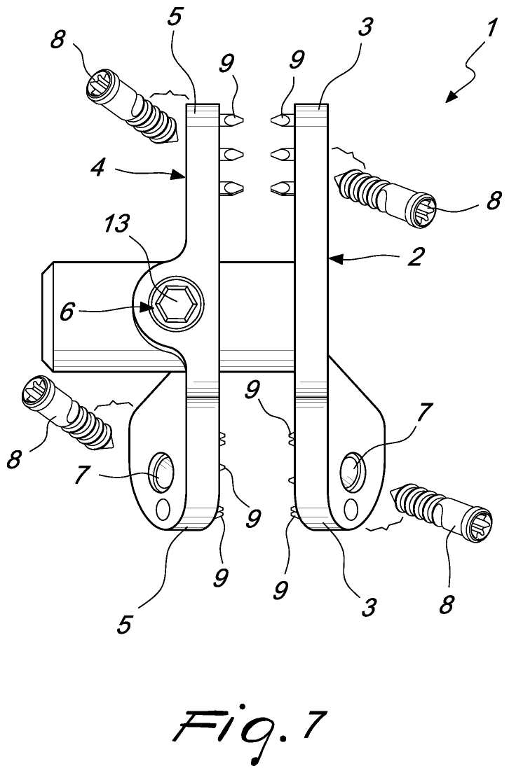

FIG. 6 is a schematic front elevation view of a fourth step, of tightening the screws inserted in the third step, for the stabilization of the prosthesis;

FIG. 7 is a schematic front elevation view of an embodiment of an interspinous-intraspinous prosthesis according to the invention which is intended to be implanted at the L5-S1 portion of the spinal column.

With particular reference to the figures, the reference numeral 1 generally designates an interspinous-intraspinous prosthesis.

The interspinous-intraspinous prosthesis 1 according to the invention comprises a clamp constituted by a first, fixed element 2 provided with at least one protruding tab 3 and by a second, movable element 4, which can move along the fixed element 2 and is provided with at least one protruding contrast tab 5.

Removable mutual locking means 6 can advantageously be interposed between the first, fixed element 2 and the second, movable element 4.

According to the invention, at least one component chosen between the tab 3 and the contrast tab 5 comprises at least one through hole 7 for the accommodation of at least one screw 8 designed to be tightened on a respective spinous process A of a vertebral body B clamped between such tab 3 and such contrast tab 5.

Each screw 8 can profitably have a variable length from 3 mm to 14 mm, according to the specific applicative requirements and the anatomical characteristics of the patient. However, the possibility is not ruled out of adopting screws 8 of different lengths in order to meet specific requirements, while remaining within the scope of protection defined by this report.

It should furthermore be noted that, conveniently, the at least one hole 7 can be inclined, with respect to the surface of the tab 3/contrast tab 5, by an angle comprised between 5° and 80°, in order to facilitate the tightening of a respective screw 8 by the operator during the implantation of the prosthesis 1 according to the invention.

In fact the operator will be in a position to easily tighten each screw 8 by acting from outside in the incision made surgically in the back of the patient: the fact that the head of each screw 8, by virtue of the inclination of the respective hole 7, is directed outward and therefore toward the operator, will make the respective tightening operations simpler.

It should be noted that each tab 3 can comprise at least one respective hole 7 which is designed to accommodate a respective screw 8: overall the interspinous-intraspinous prosthesis 1 will profitably comprise at least four screws 8 arranged in respective inclined holes 7.

In this manner the locking of the implanted prosthesis 1 will not rely solely on clamping the spinous processes A of two separate vertebral bodies B, which is achieved by virtue of the tabs 3 and the contrast tabs 5, but will also take advantage of the greater stability conferred by the presence of the screws 8 which will be anchored and screwed directly into corresponding portions of the spinous processes A of the two vertebral bodies B to be coupled.

With particular reference to an embodiment of undoubted practical and applicative interest, the at least one hole 7 can conveniently be inclined, with respect to the surface of the tab 3 and/or of the contrast tab 5, by an angle comprised between 15° and 50°.

It should furthermore be noted that the first element 2 and the second element 4 can advantageously comprise respective mutually parallel tabs 3 and contrast tabs 5, for the clamping of the spinous processes A of vertebral bodies B of the L1-L5 lumbar region.

There is also a variation of embodiment in which, instead, at least one component chosen between either the first element 2 or the second element 4 comprises respective tabs 3 or contrast tabs 5 which are inclined by 45° with respect to the contrast tabs 5 or to the tabs 3 of the at least one other component chosen between either the second element 4 or the first element 2, for the clamping of the spinous processes A of vertebral bodies B of the L5-S1 lumbo-sacral region.

In particular the tabs 3 and contrast tabs 5 located in the distal end of each element 2, 3 will be tilted by an angle comprised between 0° and 80° with respect to the tabs that are located in the proximal end, for the L5-S1 portion, in that the vertebra S1 anatomically assume a more angled form with respect to the vertebra L5.

It should furthermore be noted that at least one of the surfaces directed toward the spinous process A in the configuration for use, of either the at least one tab 3 or the at least one contrast tab 5, comprises at least one protruding tooth 9 which is designed for anchoring on the spinous process A following the tightening of the clamp (constituted by the pair of elements 2 and 4).

In reality there will be a plurality of teeth 9, set close to each other, adapted favor a better grip on the spinous processes A.

In the specific geometric structural shape of the prosthesis 1, the first, fixed element 2 profitably comprises an elongated main body 10 which is provided, at a head thereof, with the transversely protruding tabs 3. In such case the second, movable element 4 will profitably comprise a through cavity 11 the shape and dimensions of which are complementary to those of the elongated main body 10, for the slideable accommodation thereof.

Advantageously, the elongated body 10 and the cavity 11 are not circular in shape in order to prevent mutual rotations of the two elements 2, 4 when they are coupled: in this manner the two components will behave like a prismatic pair, allowing them only to slide on each other without any mutual rotation being permitted.

It should be noted that the second, movable element 4 comprises a threaded hole 12 for the accommodation of a threaded element 13, the terminal front of which abuts against the elongated main body 10.

The hole 12 and the threaded element 13 will constitute the removable mutual locking means 6 of the first element 2 and of the second element 4.

The threaded hole 12, in a specific embodiment, can have a diameter of approximately 3 mm (although the possibility is not ruled out of adopting different diameters) in order to allow the entry of a threaded element 13 of variable length between 3 mm and 5 mm (although the possibility is not ruled out of adopting different lengths), thus allowing the mutual locking together of the first element 2 and of the second element 4.

It should be noted that at least one of either the first element 2 or the second element 4 comprises at least one through slot 14 for the temporary accommodation of specific supporting tools used by the at least one operator during the implantation of the prosthesis 1 in the patient.

Similarly, at least one of either the first element 2 or the second element 4 comprises at least one through opening 15 for the temporary accommodation of specific tightening tools used by the at least one operator during the implantation of the prosthesis 1 on the patient to bring the tabs 3 closer to the contrast tabs 5, in so doing clamping the spinous processes A.

The prosthesis 1 is available in various sizes which are characterized by different heights: 3.5 mm, 4.5 mm, 5.5 mm, 6.5 mm, 8.5 mm, 10.5 mm, 12.5 mm, 14.5 mm (although the possibility is not ruled out of adopting different heights).

The screws 8 will have a preferred diameter of 3.0 mm (although the possibility is not ruled out of adopting different diameters) and are available in various lengths: 0.6 mm, 0.8 mm, 1.0 mm (although the possibility is not ruled out of adopting different lengths).

The interspinous-intraspinous prosthesis 1 according to the invention, intended in particular for the lumbar region L1-S1 of the spinal column, substantially modifies the concept of lumbar stabilization from the rear: it is a prostheses 1 for which implantation is less invasive, while still ensuring high stability by virtue of the presence of the trans-bone screws 8. The adoption of the screws 8 is, among other things, consistent with the concept of regenerative bone stimulation, by virtue of their transcortical presence, just like screws in a transpeduncular position. The prosthesis 1 according to the invention can be considered the first interspinous-intraspinous prosthesis with screws 8 offering low biological impact and certain arthrodesis.

The presence of numerous transcortical stabilization screws 8 ensures, by virtue of the corresponding positioning, that the prosthesis 1 according to the invention:
can be clamped more effectively and stably on the spinous processes (also by using teeth 9 of shorter length than those seen on conventional prostheses);
triggers arthrodesis with greater rapidity and effectiveness, for a faster periosteal reaction ensured by low injury to the periosteum during the clamping step;
is also adapted for implantation at the region of the spinal column known as L5-S1.

The entire prostheses 1, or even exclusively parts and/or components thereof, can profitably be made of titanium, alloys of titanium, steel and other metallic alloys that are biocompatible or even bioabsorbable. However, the possibility is not ruled out of adopting composite materials for specific applications.

Advantageously the present invention solves the above mentioned problems, by providing an interspinous-intraspinous prosthesis 1 that offers a high stability right from the moment of its implantation, by virtue of the simultaneous presence of the two elements 2 and 4 which clamp the spinous processes A of the two vertebral bodies B to be coupled and of the screws 8 that bite directly into the spinous process A, thus becoming anchored therein.

Conveniently the interspinous-intraspinous prosthesis 1 according to the invention enables the patient not to require long periods of immobility after the operation to implant the prosthesis 1, in that the stability thereof is superior to that of conventional prostheses and therefore favors a faster and more effective radication.

Usefully the interspinous-intraspinous prosthesis 1 according to the invention has a space occupation corresponding entirely to that of traditional prostheses.

Positively the interspinous-intraspinous prosthesis 1 according to the invention can be easily implanted with operating techniques substantially similar to those in use for conventional prostheses.

Advantageously the interspinous-intraspinous prosthesis 1 according to the invention is adapted to ensure the necessary formation of bone tissue within substantially contained times (facilitated by the mutual immobility of the two coupled vertebral bodies B, as well as by a periosteum that is preserved during the compression phase).

Positively the interspinous-intraspinous prosthesis 1 according to the invention is easily and practically implemented and is of low cost: such characteristics make the prosthesis 1 according to the invention an innovation that is safe in use.

The invention, thus conceived, is susceptible of numerous modifications and variations, all of which are within the scope of the appended claims. Moreover, all the details may be substituted by other, technically equivalent elements.

In the embodiments illustrated, individual characteristics shown in relation to specific examples may in reality be interchanged with other, different characteristics, existing in other embodiments.

In practice, the materials employed, as well as the dimensions, may be any according to requirements and to the state of the art.

The disclosures in Italian Patent Application No. 102021000030545 from which this application claims priority are incorporated herein by reference.

Where technical features mentioned in any claim are followed by reference signs, those reference signs have been included for the sole purpose of increasing the intelligibility of the claims and accordingly, such reference signs do not have any limiting effect on the interpretation of each element identified by way of example by such reference signs.

1-10. (canceled)

11. An interspinous-intraspinous prosthesis of the type comprising a clamp constituted by a first, fixed element provided with at least one protruding tab and by a second, movable element, which is configured to move along said first, fixed element and is provided with at least one protruding contrast tab, removable mutual locking means being interposed between said first, fixed element and said second, movable element, wherein at least one component chosen between said at least one protruding tab and said protruding contrast tab comprises at least one through hole for accommodation of at least one screw designed to be tightened on a respective spinous process of a vertebral body clamped between said at least one protruding tab and said protruding contrast tab.

12. The interspinous-intraspinous prosthesis according to claim 11, wherein said at least one hole is inclined, with respect to a surface of said at least one protruding tab/protruding contrast tab, by an angle comprised between 0° and 80°, in order to facilitate tightening of a respective screw by an operator during implantation of said prosthesis.

13. The interspinous-intraspinous prosthesis according to claim 12, wherein said at least one hole is inclined, with respect to the surface of said at least one protruding tab/protruding contrast tab, by an angle comprised between 5° and 80°, in order to facilitate tightening of a respective screw by the operator during the implantation of said prosthesis.

14. The interspinous-intraspinous prosthesis according to claim 11, wherein said first, fixed element and said second, movable element comprise respective mutually parallel protruding tabs and protruding contrast tabs for the clamping of the spinous processes of vertebral bodies of the L1-L5 lumbar region.

15. The interspinous-intraspinous prosthesis according to claim 11, wherein at least one component chosen between either said first, fixed element or said second, movable element comprises respective protruding tabs and protruding contrast tabs which are inclined by an angle comprised between 0° and 80° with respect to the protruding contrast tabs and protruding tabs of at least one other component chosen between either said second, movable element or said first, fixed element, for the clamping of the spinous processes of vertebral bodies of the L5-S1 lumbo-sacral region.

16. The interspinous-intraspinous prosthesis according to claim 15, wherein at least one of surfaces directed toward the spinous process in a configuration for use, of either said at least one protruding tab or said at least one protruding contrast tab, comprises at least one protruding tooth which is designed for anchoring on said spinous process following the tightening of said clamp.

17. The interspinous-intraspinous prosthesis according to claim 11, wherein:
said first, fixed element comprises an elongated main body which is provided, at a head thereof, with said at least one protruding tab;
said second, movable element comprises a through cavity a shape and dimensions of which are complementary to those of said elongated main body, for accommodation thereof.

18. The interspinous-intraspinous prosthesis according to claim 17, wherein said second, movable element comprises a threaded hole for accommodation of a threaded element, a terminal front of which abuts against said elongated main body, said hole and said threaded element constituting said removable mutual locking means of said first, fixed element and of said second, movable element.

19. The interspinous-intraspinous prosthesis according to claim 11, wherein at least one of either said first, fixed element or said second, movable element comprises at least one through slot for temporary accommodation of supporting tools used by the at least one operator during the implantation of said prosthesis in a patient.

20. The interspinous-intraspinous prosthesis according to claim 11, wherein at least one of either said first, fixed element or said second. movable element comprises at least one through opening for temporary accommodation of tightening tools used by the at least one operator during the implantation of said prosthesis in a patient.
